(19) United States
(12) Patent Application Publication
(10) Pub. No.: US 2024/0139484 A1
(43) Pub. Date: May 2, 2024

(54) Test Equipment for Microneedle Systems, and a Test System and a Method for Testing a Microneedle Application

(71) Applicant: LTS Lohmann Therapie-Systeme AG, Andernach (DE)

(72) Inventors: Sebastian Scherr, Neuhäusel (DE); Miriam Britten, Andernach (DE)

(21) Appl. No.: 18/272,678

(22) PCT Filed: Jan. 18, 2022
(86) PCT No.: PCT/EP2022/050930

§ 371 Date: Jul. 17, 2023

(30) Foreign Application Priority Data
Jan. 18, 2021 (DE) 10 2021 100 908.1

(51) Int. Cl. A61M 37/00; G01D 21/02

(52) U.S. Cl. CPC A61M 37/0015; G01D 21/02; A61M 2037/0061

(57) ABSTRACT

Described is a test device for microneedle systems having a microneedle receptacle for receiving a microneedle system to be tested, an application device, and a movement device including a punch for the application in motion of the microneedle system to be tested in the application device. A test system with a test device of this kind is also described. Lastly, described is a method for testing a microneedle application.

### CROSS-REFERENCE TO RELATED APPLICATIONS

This application is the United States national phase of International Application No. PCT/EP2022/050930 filed Jan. 18, 2022, and claims priority to German Patent Application No. 10 2021 100 908.1 filed Jan. 18, 2021, the disclosures of which are hereby incorporated by reference in their entireties.

### BACKGROUND OF THE INVENTION

### Field of the Invention

The invention relates to a test device for microneedle systems, a test system for testing a microneedle application and to a method for testing a microneedle application.

Beside classic forms of active ingredient administration, such as for example swallowing medicaments, injecting medicine or the like, there are concepts for an application of active ingredients via the skin. For example, active ingredients can be introduced into the skin using microneedles.

Microneedle arrays, also referred to as microarrays, have a plurality of microneedles that are typically arranged on a support element, such as a patch, a plaster or the like, or connected to a support element. Such microarrays include a high number of microneedles, for example 100 to 1000 needles per cm 2. The needles have a short length, so that upon being pressed into a patient's skin, the needles penetrate the skin only so far that nerves and blood vessels make no contact with needle tips, if possible. The microneedles comprise an active ingredient or a medicament. The corresponding active ingredient may be provided on a surface of the needles or be provided in the needles. It is preferred that the needles are made of a material dissolving in the skin.

### Description of Related Art

The application of microneedles, in particular of microarray patches, into the skin depends on many factors. Among these are the number of needles, the needle geometry or the micro needle material used. Moreover, the structure of the skin also plays a role. For example, a restoring force from the skin acts against the microneedles applied. This restoring force varies depending on the skin or the skin type. Also the application as such is of particular importance. For example, microneedles can be applied manually, e.g., by being pushed in using a finger or a hand, or by means of applicators. An applicator for applying microneedles is disclosed e.g. in PCT/EP 2020/073670.

Presently, there are no devices or methods for testing or checking the application of microneedles, in particular no standardized devices or methods.

### SUMMARY OF THE INVENTION

It is an object of the invention to provide a test device, a test system and a method for microneedle systems for the, in particular standardized, testing of a microneedle application.

According to the invention, the object is achieved with a test device as described herein, a test system as described herein and a method as described herein.

The test device for microneedle systems according the invention is, in particular, a test device for microarray patches. The test device comprises a microneedle receptacle for receiving a microneedle system to be tested. Further, the test device comprises an application device. The application device is configured to apply the microneedle system into the application device. In other words, the application device is, in particular, a kind of target object for the application of the microneedle system. The application device preferably comprises a skin model or is configured to receive a skin model. When the application device is designed to receive a skin model, it is preferred that the application device comprises, in particular consists of a fixing device, such as e.g. a clamping means, for receiving the skin model. The application device is preferably two-dimensional. It is preferred that the skin model can comprise, in particular consist of human or animal skin, in particular ex vivo skin. On the other hand, it is possible that the skin model is an artificial one. In addition, the test device comprises a movement device for the application movement of the microneedle systems to be tested into the skin model. The movement device is in particular an acceleration device for the acceleration of the microneedle system. The movement device comprises a punch. The punch is configured to act on the microneedle system. In particular, the punch comprises a punch body. It is preferred that the punch has a punch surface, preferably on the punch body, for acting on the microneedle system. In other words, the punch surface is an acting surface or a working surface for the microneedle system. The punch surface is preferably designed smaller, equal in size, or larger than an application surface of the microneedle system to be tested. The application surface of the microneedle system to be tested is in particular the surface opposite the microneedles of the microneedle system, preferably the surface of a patch. In a preferred embodiment, the test device comprises a housing in which the movement device is arranged. It is preferred that the movement device and/or the punch are movable relative to the housing. The movement device and/or the punch are moveable, in particular, within the housing, preferably coaxially within the housing. It is possible that the punch has a punch protrusion which forms the punch surface, said protrusion being in particular connected integrally with the stamp body and being cylindrical in shape.

It is preferred that the microneedle receptacle and the stamp are connected, in particular designed as one piece. In a preferred embodiment, the punch, in particular the punch surface, is adapted to be connected to the microneedle system to be tested. It is possible that the connection between the punch and the microneedle system is a one-piece connection, also referred to as integral. It is particularly preferred that the punch comprises an in particular positive and/or non-positive and/or bonded fixation for connection to the microneedle system. For example, it is possible that the fixation is of an adhesive and/or clamping type. On the other hand, it is conceivable that the punch and the microneedle system are designed as separate elements. For example, the microneedle system can be arranged on the application device, in particular on the skin model, in a non-applied state and can be applied by acting thereon via the stamp. When acting thereon, preferably an in particular pulse-like contact occurs between the punch and the microneedle system.

It is preferred that the punch is connected to the movement device. The punch and the movement device are preferably connected in a rigid manner or can be connected in a rigid manner. In this context, rigid preferably means that the punch and the movement device are immovable relative to each other. It is possible that the movement device is formed by the punch. It is possible that the stamp is formed as one piece, also referred to as integrally, with the movement device. On the other hand, the punch can be designed to be exchangeable. For an exchangeable design, it is preferred that the punch and/or the movement device comprise a preferably positive or non-positive fixation device for connecting the punch to the movement device. It is possible that the punch comprises a punch fixation. The punch fixation is arranged in particular opposite the punch surface. Preferably, the punch fixation is a positive fixation, for example, for clamping the punch in the movement device which preferably comprises a clamping device. The punch fixation is in particular cylindrical with a preferably circular or square cross section. It is particularly preferred, that the punch fixation is connected to the punch body to form one piece with the same, i.e. integrally.

In a preferred embodiment, the punch, in particular the punch body, has a punch mass. The punch mass serves to accelerate the punch. Preferably, the mass of the punch and the velocity generated by the movement device generate a pulse, in particular for application. The punch mass can preferably accelerate the microneedle system for application and/or generate a retention force on the microneedle system, in particular upon application. As a result, it is preferred that the punch mass causes the microneedle system to be applied and/or retained in an applied state in the application device, in particular in the skin model. The punch mass is preferably 50 300 g. It is preferred that the test device is configured such that a mass of 200-500 g acts upon application. This mass is in particular an applied mass which is at least partially formed by the punch mass.

In a preferred embodiment, the punch, in particular the punch body, is cylindrical or conical in shape. It is particularly preferred that the conical shape is designed as a truncated cone. The cross section of the cylindrical or conical shape may, for example, be circular, oval, triangular, rectangular, in particular square. Thus, a rectangular, in particular square design preferably is a pyramid shape, preferably embodied as a frustum of a pyramid. It is preferred that the conical shape tapers towards the punch surface. It is particularly preferred that the punch surface corresponds to a stump surface of the truncated cone.

In a preferred embodiment, the test device comprises an actuator. The actuator is configured to exert force on the movement device, in particular on the punch. The exertion of force is preferably designed to accelerate the punch and/or to generate a retention force at the punch. Thereby, it is in particular advantageously possible to accelerate the microneedle system and to apply the microneedle system and/or retain the same in an applied state. The actuator is in particular mechanical and/or electric. The actuator preferably comprises, in particular consists of a motor and/or an energy store, preferably a spring. If the movement device is the punch, it is e.g. possible that, in particular starting from an initial situation, the punch is movable with respect to at least a part of the actuator. Further, it is preferably possible that the punch can be connected to the actuator in an exchangeable manner via a punch fixation, e.g. via a positive and/or a non-positive connection. If the actuator generates a retention force, it is preferred that the retention force is greater than the weight force of the punch mass.

It is preferred that the test device comprises a guide, in particular a linear guide, for a guided movement of the movement device, in particular the punch and/or the microneedle system. Due to the guide, it is in particular advantageously possible to perform a guided application of the microneedle system into the application device, in particular into the skin model. Specifically, the actuator and/or the movement device comprises the guide.

In a preferred embodiment, the test device comprises a spacer to define a distance between the punch and the application device, in particular between the punch and the skin model. The spacer preferably defines a distance between the punch in the initial position and/or in the deflected position and the application device, in particular the skin model. It is particularly preferred that the distance exists between the punch surface and the application device, in particular between the punch surface and the skin model. It is preferred that the spacer is variable so that a variable distance can be set. The spacer preferably comprises, in particular consists of an adjusting nut. The adjusting nut is preferably hollow, it being preferred that the punch is arranged, in particular coaxially, in the adjusting nut. The adjusting nut allows to adjust, in particular, a clearance. The spacer advantageously allows to define a minimum distance between the punch and the application device, in particular the skin model, so that the penetration depth of the microneedle system into the application device, in particular the skin model. It is preferred that the spacer is designed such that a distance of 0-10 mm, preferably 1-5 mm, exists between the punch in the initial position and the application device, in particular the skin model. Specifically, the spacer is designed such that a distance of 0-10 mm, preferably 1-5 mm, exists between the punch in the deflected position and the application device, in particular the skin model.

It is preferred that the test device comprises a sensor device. The sensor device comprises in particular at least one sensor. The sensor device is preferably designed for detecting an application of the microneedle system into the application device, in particular the skin model. The at least one sensor is preferably connected, in particular rigidly, to the punch and/or the application device.

It is preferred that the sensor device, in particular the at least one sensor, is designed for detecting a penetration depth of the microneedle system into the application device, in particular the skin model. As an alternative or in addition, a design for detecting an application force is preferred. Application force means, in particular, a force between the microneedle system and the application device, in particular the skin model, or between the punch and the application device, in particular the skin model. It is preferred that the sensor device detects a pressure. Preferably, the sensor device is configured to detects an application force of the punch and/or a restoring force of the application device, in particular of the skin model. The restoring force results in particular from the characteristics of the skin model, e.g. a resistance of the skin model against the application of the microneedle system. For example, an elasticity of the skin model can result in a restoring force against the applied microneedles of the microneedle system and/or in a restoring force against the microneedle system as such. Preferably, the sensor device detects once, several times or continuously.

The sensor device comprises in particular one or more of the following sensors: a piezoelectric sensor, an OCT sensor, a force transducer or an acceleration sensor. If the sensor device comprises at least one piezoelectric sensor, it is preferred that the same is connected to the application device and/or the skin model. If the sensor device comprises at least one OCT sensor, it is preferred that the same is connected to the application device. If the sensor device comprises at least one force transducer, it is preferred that the same is connected to the application device and/or the skin model and/or the punch, preferably the punch surface. If the sensor device comprises at least one acceleration sensor, it is preferred that the same is connected to the punch. The force transducer is in particular a magnetic force transducer which preferably comprises at least one coil.

The test device preferably comprises a frame, in particular with a base plate. By means of the base plate, the test device can be arranged e.g. on a table or the ground. Thus, the test device is in particular a test stand. It is preferred that the base plate comprises the application device or is connected to the same. The test device, in particular the frame, preferably comprises a guide device for guiding the movement device, in particular the housing of the movement device, with respect to the application device. It is preferred that the guide device is fixable and/or adjustable, so that the movement device, in particular the housing of the movement device, is adjustable relative to the application device. Preferably, the guide device is a linear guide device. It is preferred that the guide device comprises a fixation device, which comprises, in particular, a clamping lever, for a variable movement fixation of the movement device, in particular the housing of the movement device.

In a preferred embodiment, the guide device comprises a damper and/or a spring. The spring is on particular a gas spring or a hydraulic spring. The guide device with a damper and/or a spring is in particular designed and/or arranged such that the movement device, preferably the housing of the movement device, is sprung and/or supported. It is particularly preferred that the damper and/or the spring are designed to compensate the weight of the movement device, preferably of the housing including the movement device. Specifically, it is advantageously implemented that the movement device, preferably the housing of the movement device, can be moved practically in a force-free manner.

Preferably, the test device comprises a control device which in particular transmits data and/or current. The control device is configured in particular to control the actuator. It is preferred that the control device includes a control software. The control device is configured in particular to define the acceleration and/or the velocity of the movement device, in particular via the actuator. It is further possible that the control device sets a retention force, in particular for a definable time.

The test system for testing microneedle applications according to the invention is in particular a test system for testing applications of microarray patches. The test system comprises a test device with one or a plurality of the test device described above. The test system further comprises a microneedle system, in particular a microarray patch. The microneedle system is connected to, in particular received in the microneedle receptacle. In addition, the test system comprises a skin model preferably including human or animal skin. The skin model is connected to, in particular received in the application device.

The method for testing microneedle applications according to the invention is in particular a method for testing applications of microarray patches. The method steps described below are preferably executed in the order mentioned below. A first step of the method consists in accelerating a microneedle system comprising microneedles. The microneedle system preferably is a microarray patch. A second step consists in applying the microneedles of the microneedle system into a application device which preferably comprises a skin model. The application of the microneedles is effected in particular by the acceleration. A third step consists in detecting, in particular measuring, the application by means of a sensor device. The sensor device is configured in particular like the sensor device described above for the test device. The sensor device preferably comprises at least one sensor. It is particularly preferred that a penetration depth and/or an application force is detected when the application is detected. In particular, the sensor device is configured to detect the penetration depth and/or the application force. In this context, application force means in particular a force prevailing between the microneedles of the microneedle system and the application device, in particular the skin model, and/or between the microneedle system and the application device, in particular the skin model.

The method is preferably executed with test device having one or more features of the test device described above or a test system having one or more features of the test system described above.

The test device according to the invention, the test system according to the invention and/or the method according to the invention are advantageous in particular because application tests with microneedle systems can be standardized. Preferably, the knowledge about application parameters thus obtained can be used in designing applicators for use with microneedle systems. In this manner, it can, for example, be ensured at a later time that the application of the microneedle systems works safely and reliably and no complications or wrong applications occur.

### BRIEF DESCRIPTION OF THE DRAWINGS

In the following, the invention is described in more detail by means of preferred embodiments with reference to the accompanying drawings.

In the drawings:

FIG. 1 is a schematic sectional side view of an embodiment of a test system according to the invention with an embodiment of a test device according to the invention

FIG. 2 is a schematic sectional side view of a further embodiment of a test system according to the invention with a further embodiment of a test device according to the invention,

FIGS. 3a-3d are schematic sectional side views of embodiments of punches of the test device,

FIG. 4a is a perspective view of a further embodiment of a test system according to the invention with a further embodiment of a test device according to the invention, and

FIG. 4b shows the illustration in FIG. 4a with a transparent view of a detail.

### DESCRIPTION OF THE INVENTION

In the FIGS., similar or identical components or elements are identified by the same reference numerals. In particular in the interest of improved clarity, preferably elements already identified are not provided with reference numerals in all FIGS.

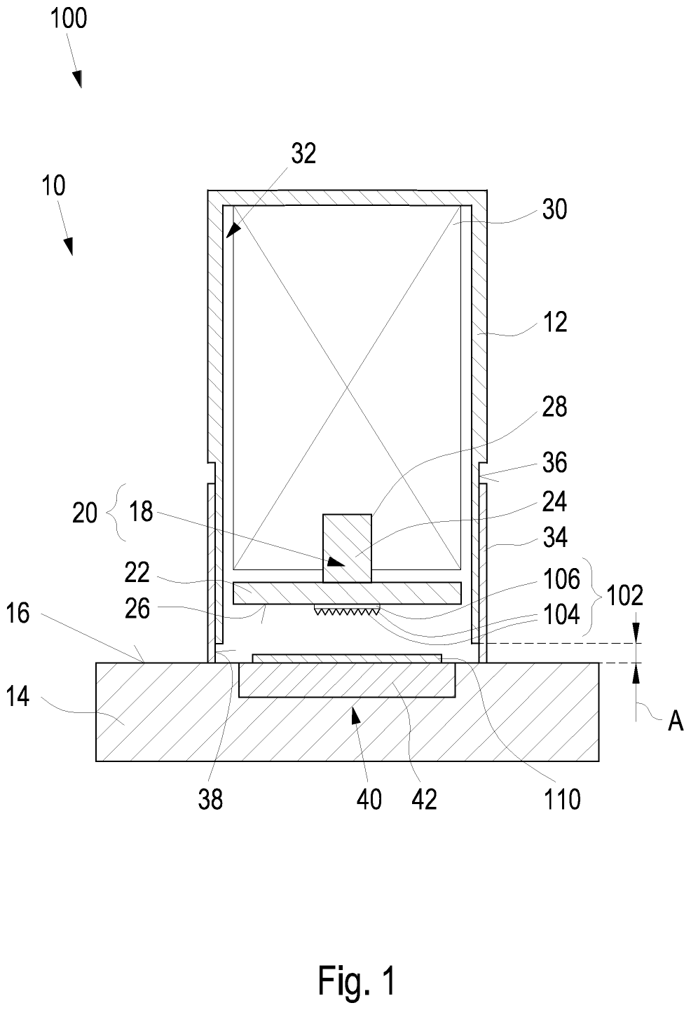

FIG. 1 shows a test system 100 with a test device 10, a microneedle system 102 and a skin model 110.

The test device 10 comprises a housing 12 having a hollow cylindrical shape closed on one side. In the area of the open end, the hollow cylindrical shape has a protrusion 36 with outer threads. An adjusting nut 34 is screwed on the outer threads, the nut having corresponding inner threads on the inner circumference 38. By turning the adjusting nut 34, a distance A of the housing 12 to the surface 16 of the application device 14 can be adjusted.

An actuator 30 is fixedly, i.e. immovably, in particular integrally connected to the closed end of the housing 12. The actuator 30 is arranged in particular coaxially inside the housing 12. The actuator 30 is in particular an electric and/or a mechanic actuator. For example, the actuator 30 can comprise or consist of a spring and/or a motor. The actuator 30 is configured to move, in particular accelerate a movement device 20. For movement, the actuator 30 acts on the movement device 20.

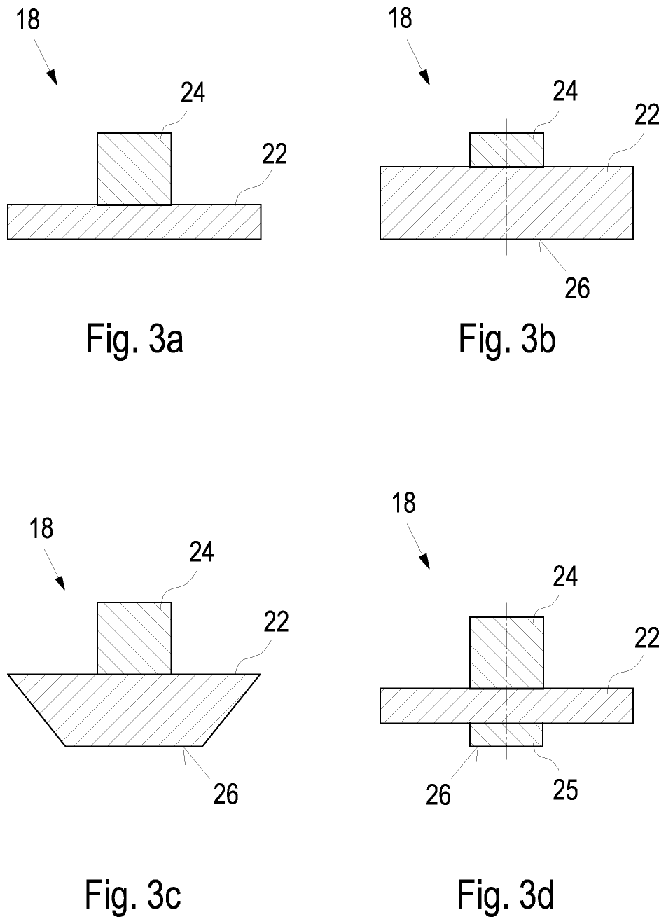

In the embodiment illustrated, the movement device 20 merely comprises, i.e. consists of a punch 18. The punch 18 comprises a punch body 22, as well as a punch fixation 24 connected with the same, in particular integrally. The punch 18 is connected to the punch receptacle 28 of the actuator 30 via the punch fixation 24. In this case, the connection between the actuator 30 and the punch 18 is designed to be movable, so that the punch 18 is movable relative to the actuator 30. The punch 18 is thus preferably displaceable in the punch receptacle 28 of the actuator 30, in particular linearly. Upon acceleration by the actuator 30, the punch 18 moves relative to the actuator 30 and the housing 12. It is possible that a motor and/or a spring of the actuator 30 acts on the stamp fixation 24 in order to cause a movement of the punch 18. The punch fixation 24 and the punch receptacle 28 allow, in particular, to exchange the punch 18 and thus, for example, provide different punches (see e.g. FIGS. 3a-3d).

A microneedle system 102 is connected to the punch surface 26 of the punch 18. Here, the connection is in particular an adhesive connection. Thus, a microneedle receptacle is realized through this adhesive connection.

The microneedle system 102 comprises a support element 106, as well as a plurality of microneedles 104 connected thereto in particular integrally. In particular, the microneedle system 102 is a microarray patch.

The test device 10 is arranged on an application device 14, a spacing having the distance A being established by the spacer having an adjusting nut 34. A skin model 110 is connected to the application device 14. As illustrated, this connection between the application device 14 and the skin model 110 is in particular an adhesive connection, the skin model 110 sticking on the upper side 16 of the application device 14.

A sensor device 40 is connected to the application device 14. As illustrated, the sensor device 40 is received in the application device 14. As illustrated, the sensor device 40 comprises a sensor 42. The sensor 42 may for example be an OCT sensor or a piezoelectric sensor. The sensor 42 is configured to sense the skin model or detect an application of the microneedle system 102 into the skin model 110. Here, it is possible that the skin model 110 is connected, in particular directly, to the sensor 42. On the other hand, it is also possible that the sensor 42 and the skin model 110 are spaced apart from each other.

Upon acceleration of the punch 18, the microneedle system 102 connected to the punch 18 is accelerated. This acceleration of the microneedle system 102 causes an application of the microneedle system 102 into the skin model 110.

The application can be detected by the sensor device 40. It is possible, for example, to detect a penetration depth e.g. by means of an OCT sensor. As an alternative or in addition, it is possible, for example, to detect an application force, in particular by means of a piezo-electric sensor. The sensor device 40 can also be used to detect, for example, a restoring force of the skin model 110 against the application.

For example, a clearance from the punch surface 26 can be adjusted variably via the adjustable distance A. Thus, it is possible to adjust different application depths.

As an alternative to the embodiment in FIG. 1, it is possible that the microneedle system 102 is not connected to the punch 18, but is arranged on the skin model 110, for example. Also in such an embodiment, an application of the microneedle system 102 into the skin model 110 is effected by an acceleration of the punch 18, this application being performed by a pulse-like impact of the punch 18 on the microneedle system 102. A retention of the microneedle system 102 via the punch 18 is possible.

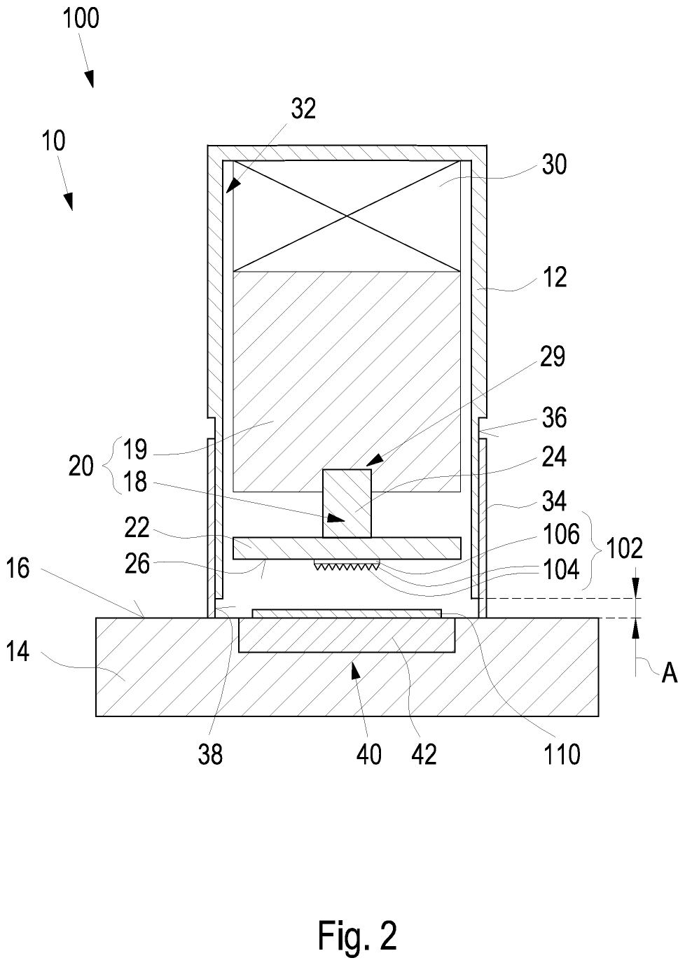

The embodiment in FIG. 2 corresponds substantially to the embodiment in FIG. 1. Different from FIG. 1, the movement device 20 in the embodiment in FIG. 2 comprises a punch 18 and a punch receptacle 19. The punch 18 is connected to the punch receptacle 19 via the punch fixation 24. As illustrated, the connection between the punch 18 and the punch fixation 24 is realized by a positive connection between the punch fixation 24 and a recess 29 in the punch receptacle 19. As an alternative or in addition, the connection between the actuator 30 and the punch 18 can also be made, for example, via a non-positive connection, e.g. a screw. Upon acceleration by the actuator 30, the movement device 20 moves relative to the housing. In this regard, it is possible that a motor and/or a spring of the actor 30 can act on the punch receptacle 19 in order to move the movement device 20 with the punch 18. The punch fixation 24 and the recess 29 allow to exchange the punch 18 and thus, for example, provide different punches (see e.g. FIGS. 3a-3d).

FIGS. 3a-3d show different punch designs of the punch 18.

FIG. 3a illustrates in particular a punch design according to the embodiment of the punch 18 in FIG. 1. The punch 18 comprises a punch body 22 and a punch fixation 24. The punch body 22 is preferably cylindrical. The punch body 22 can comprise a punch mass with a weight. An application force for the application of the microneedle system 102 can be varied by means of different punch masses.

FIG. 3b illustrates another design of the punch 18, the punch body 22 having a greater punch mass than the design of FIG. 3a. Thus, when compared to the design of FIG. 3a, a greater application force and/or retention force can be implemented. Similar to the illustration in FIG. 1, the punch surface 26 of the punch 18 in FIG. 3b is designed such that it covers the entire application surface of a microneedle system 102. Thus, a full-surface application of a microneedle system 102 is possible through the punch surface 26.

FIG. 3c illustrates another design of the punch 18. In this case, the punch body 22 is frustoconical. Starting from the punch fixation 24, the punch body 22 tapers to the punch surface 26. The punch surface 26 is thus smaller than the punch surface 26 of FIG. 3b. Accordingly, it is possible, for example, that the punch surface covers only a part of the application surface of a microneedle system 102. Thus, it is possible to perform a punctual application by an application with the punch 18 of FIG. 3c. In this manner, it is possible, for example, to simulate a manual application of a microneedle system 102 pressed by means of a finger.

FIG. 3d illustrates another design of a punch 18. The punch in FIG. 3d corresponds essentially to the punch 18 in FIG. 3a. In contrast with the punch 18 in FIG. 3a, the punch 18 of FIG. 3d comprises a punch protrusion 25 connected, in particular integrally, to the punch body 22, so that the punch surface 26 is offset. The punch protrusion 25 is in particular cylindrical in shape. The punch surface 26 of the punch protrusion 25 is, for example, smaller, equal in size or larger than an application surface of a microneedle system 102.

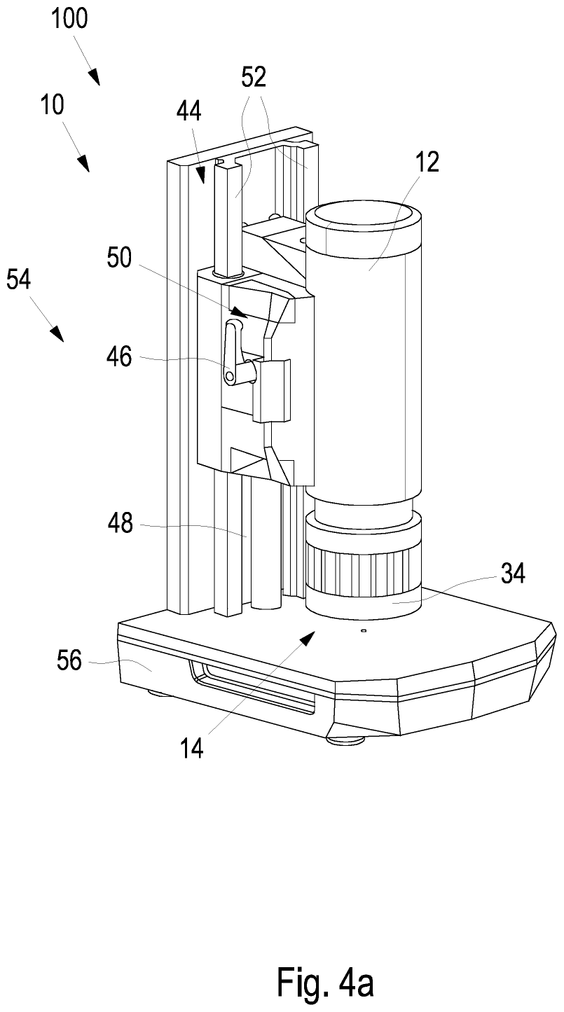

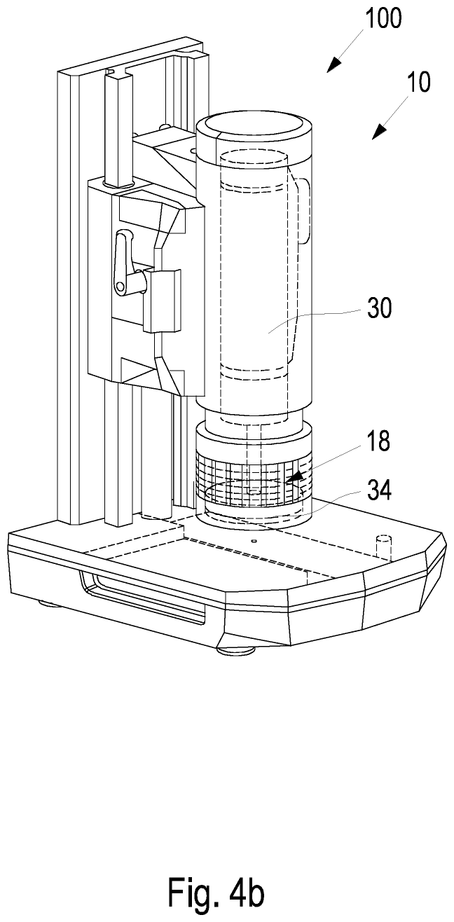

FIGS. 4a-4b illustrate a further embodiment of a test system 100 with a test device 10.

The test device 10 has a frame 54 with a base plate 56 and a guide device 44. The base plate 56 is connected to an application device 14 on which a skin model 110 can be arranged. The guide device 44 comprises rails 52 guiding a slide 50. The housing 12 can be displaced via the rails 52 and the slide 50. By means of a fixing device 46 comprising or consisting of, in particular, a clamping lever, a fixation can be made between the slide 50 and the rails 52, so that a height of the housing 12 is adjustable. A spring device 48, preferably comprising a gas spring, supports the slide 50 and the housing 12, in particular in a weight-neutral manner.

In FIG. 4b, the interior of the housing 12 and the interior of the adjusting nut 34 are visible, since these elements are shown in a transparent manner. Similar to the embodiment in FIG. 1, an actuator 30 is arranged in the housing 12 to accelerate a punch 18. A distance can be adjusted via the adjusting nut 34.

1. A test device for microneedle systems, in particular for microarray patches, comprising
a microneedle receptacle for receiving a microneedle system to be tested,
an application system preferably including a skin model or configured to receive a skin model, and
a movement device comprising a punch for the application movement of the microneedle system to be tested into the application device.

2. The test device according to claim 1, wherein the microneedle receptacle is connected to the punch, in particular integrally.

3. The test device according to claim 1, wherein the punch is configured to be exchangeable or integrally with the movement device.

4. The test device according to claim 1, wherein the punch has a punch mass, in particular between 50 and 300 g, for the acceleration of the microneedle system and/or for the generation of a retention force at the microneedle system.

5. The test device according to claim 1, wherein the punch has the shape of a cylinder, or the shape of a pyramid, preferably a frustum of a pyramid, or the shape of a cone, preferably a truncated cone.

6. The test device according to claim 1, characterized by an electrical and/or a mechanical actuator for the acceleration of the punch and/or the generation of a retention force at the punch.

7. The test device according to claim 1, characterized by a preferably variably adjustable spacer for defining a distance between the punch and the application device and/or the skin model.

8. The test device according to claim 1, characterized by a sensor device for detecting an application of the microsystem into the application device, in particular into the skin model, said sensor device preferably comprising at least one sensor.

9. The test device according to claim 8, wherein the sensor device is configured to detect a penetration depth of the microneedle system into the application device and/or to detect a force between the microneedle system and the application system.

10. The test device according to claim 8, wherein the sensor device comprises:
a piezoelectric sensor which is in particular connected to the application device; and/or
an OCT sensor which is in particular connected to the application device; and/or
a preferably magnetic force transducer which is in particular connected to the application device and/or the punch.

11. The test device according to claim 1, characterized by a preferably fixable guide device for guiding the movement device relative to the application device.

12. A test system for testing microneedle applications, in particular a microarray patch application, comprising
a test device according to claim 1,
a microneedle system, in particular a microarray patch, and
a skin model, preferably including human and/or animal skin,
wherein the microneedle system is connected to the microneedle receptacle, and
wherein the skin model is connected to the application device.

13. A method for testing a microneedle application, with a test device according to claim 1, comprising the steps of:
accelerating a microneedle system comprising microneedles,
applying the microneedle system into an application device which preferably includes a skin model, and
Detecting the application, in particular a penetration depth and/or an application force, by means of a sensor device preferably comprising at least one sensor.

14. A method for testing a microneedle application with a test system according to claim 12, comprising the steps of:
accelerating a microneedle system comprising microneedles,
applying the microneedle system into an application device which preferably includes the skin model, and
detecting the application, in particular a penetration depth and/or an application force, by means of a sensor device preferably comprising at least one sensor.
